(19) United States
(12) Patent Application Publication
(10) Pub. No.: US 2009/0207968 A1
(43) Pub. Date: Aug. 20, 2009

(54) DUAL X-RAY TUBE GATING

(75) Inventor: Michael Grass, Hamburg (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/305,462

(22) PCT Filed: Jun. 13, 2007
(86) PCT No.: PCT/US07/71098

§ 371 Date: Dec. 18, 2008

Related U.S. Application Data
(60) Provisional application No. 60/805,520, filed on Jun. 22, 2006.

(51) Int. Cl. A61B 6/00

(52) U.S. Cl. 378/9

(57) ABSTRACT

A computed tomography system includes at least a first x-ray source (14) that continuously emits radiation through an imaging region (22) while rotating about the imaging region (22) during a data acquisition cycle and at least a second x-ray source (14) that periodically emits radiation through the imaging region (22) while rotating about the imaging region (22) during the data acquisition cycle. A first set of detectors (24) detects projection radiation corresponding to the at least first x-ray source (14) and generates first projection data indicative of the detected radiation, and a second set of detectors (24) detects projection radiation corresponding to the at least second x-ray source (14) and generates second projection data indicative of the detected radiation. A reconstruction system (32) that reconstructs the first projection data to generate a set of images, the second projection data to generate a set of images, and/or a combination of the first and second projection data to generate another set of images.

The present application relates to medical imaging systems. It finds particular application to computed tomography (CT) and, more particularly to multi-tube gating techniques.

The x-ray tubes in a conventional multi-tube CT imaging system can be concurrently driven such that both tubes simultaneously emit radiation through a common imaging region. When concurrently driving the tubes as such, the imaging system can provide greater temporal resolution and faster data acquisition time relative to a single tube system. For example, a system with two tubes that are angularly displaced about 90 degrees from each other along the rotation axis can acquire the same data as a single tube system in about half the time. In another example, using such system for cardiac CT, data acquisition over a fraction of a 180 degree gantry angle detects enough data for a 180 degree reconstruction.

A consequence of concurrently irradiating a patient with multiple x-ray tubes is an increase in patient dose (e.g., by a factor of about two with a dual source system). Such dose increase can be reduced through x-ray tube gating techniques that concurrently turn the x-ray tubes “on” only during one or more desired sampling periods of each data acquisition cycle and turn the x-ray tubes “off” outside of these sampling periods. For instance, with cardiac CT applications, prospective ECG gating can be used to turn the x-ray tubes “on” during a window around a desired cardiac phase. The x-ray tubes are turned “off,” or emit little to no radiation outside of this window.

Although gating the tubes reduces patient dose, it also reduces the amount of information collected during a data acquisition cycle. For instance, if the tubes are gated to only one cardiac phase, the detected radiation reconstructs to generate one image in one phase. Neither four-dimensional information (e.g., three-dimensional images viewed over of time) nor information about the other cardiac phases can be derived from the detected information. In addition, since the tubes are simultaneously emitting radiation, each detector also detects cross scatter radiation, and cross scatter radiation can severely deteriorate the signal-to-noise ratio and introduce artifact into the reconstructed image.

Present aspects of the application provide a new and improved x-source tube gating technique that overcomes the above-referenced problems and others.

In accordance with one aspect, a computed tomography system includes at least two x-ray sources, corresponding detectors, and a reconstruction system. A first x-ray source continuously emits radiation and a second x-ray source periodically emits radiation during a data acquisition cycle. A first set of detectors detects projection radiation corresponding to the first x-ray source and generates first projection data indicative of the detected radiation, and a second set of detectors detects projection radiation corresponding to the second x-ray source and generates second projection data indicative of the detected radiation. A reconstruction system reconstructs the first projection data to generate a first set of images, the second projection data to generate a set second of images, and/or a combination of both data acquisitions to generate another set of images.

The invention may take form in various components and arrangements of components, and in various steps and arrangements of steps. The drawings are only for purposes of illustrating the preferred embodiments and are not to be construed as limiting the invention.

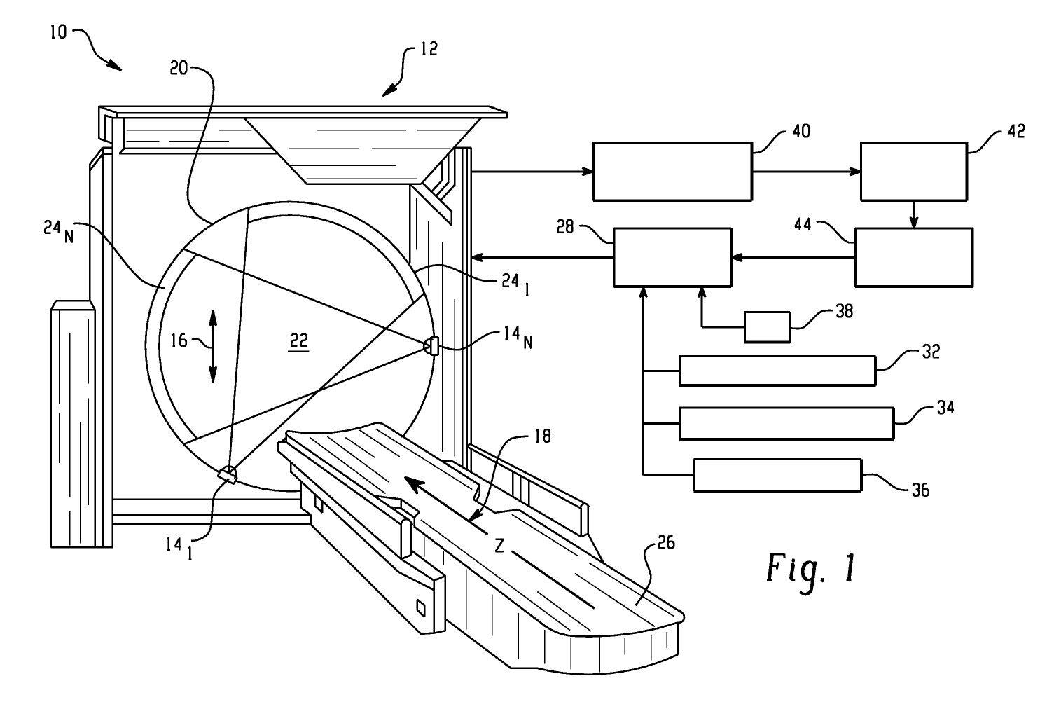

FIG. 1 illustrates a multi-source medical imaging system that employs an x-ray source gating technique to acquire different resolution data during each data acquisition cycle.

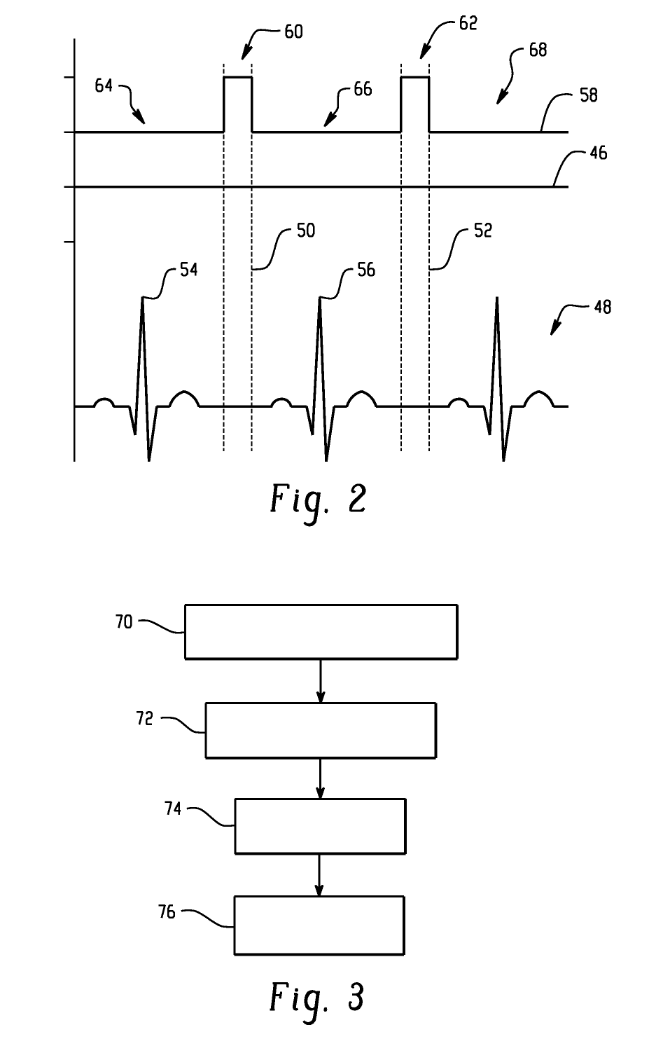

FIG. 2 illustrates an exemplary technique for gating the multiple x-ray sources with an ECG signal.

FIG. 3 illustrates an exemplary method for gating the multiple x-ray sources of a multi-source medical imaging system.

With reference to FIG. 1, a medical imaging system 10 is illustrated. The medical imaging system 10 includes multiple x-ray sources and can employ an x-ray source gating approach that gates different x-ray sources such that one or more of the x-ray sources continuously emits radiation during a data acquisition cycle while at least one other x-ray source periodically emits radiation during desired sampling periods during the same data acquisition cycle. In one instance, the medical imaging system 10 can be used in connection with cardiac CT applications. In this instance, the gating can be controlled through techniques such as ECG gating, kymogram gating, or any other sensor being able to detect the motion of the target of imaging in a prospective or retrospective (provided that information from a pre-scan is available) manner. With cardiac CT applications, such gating can be used to acquire data of different temporal, spatial and contrast resolution from different x-ray sources. For example, in one instance, at least one x-ray source can be used to acquire relatively higher resolution data and at least one x-ray source can be used to acquire relatively lower resolution data. The lower resolution data can be used to reconstruct lower resolution images of the individual cardiac phases and/or four-dimensional information such as a series of three-dimensional images over time. Such images/information can be used to monitor the dynamics of the heart muscle during a cardiac cycle and/or other observations made via lower resolution images. The higher resolution data can be used to reconstruct higher resolution images of a cardiac phase (e.g., for coronary artery imaging).

The medical imaging system 10 includes a scanner 12 having N x-ray sources 141, 14_{N} (collectively referred to herein as x-ray sources 14), wherein N is an integer greater than one. The x-ray sources 14 are positioned at an angular offset (e.g., 60, 90, 120, etc. degrees) with respect to each other within an axial or transverse plane 16 and orthogonal to a longitudinal or z-axis 18. In one instance, the x-ray sources 14 are disposed about a rotating gantry 20. Rotating the gantry 20 about an imaging region 22 rotates the x-ray sources 14 about the imaging region 22. In another instance, the x-ray sources 14 are rotated about the imaging region 22 via other techniques such as electronically deflecting an e-beam. During scanning, one or more of the x-ray sources 14 continuously and/or periodically emits radiation through the imaging region 22.

The scanner 12 further includes N sets of detectors 24₁, 24_{N} (collectively referred to herein as detectors 24). Each set of the detectors 24 subtends an angular arc opposite one of the x-ray sources 14 to define the imaging region 22 therebetween. In one instance, each detector within each set of detectors 24 rotates with and corresponds to a particular one of the x-ray sources 14 (e.g., with a third generation system). In another instance, the detectors within each set of detectors 24 reside at angular locations and, at any moment in time, are determined by the angular position of the x-ray source 14 (e.g., with a fourth generation system). Each detector within each set of detectors 24 detects radiation from actively emitting x-ray sources 14.

It is to be appreciated that in one instance the detectors 24 may have different sizes, resolution, shape, etc., the sources 14 may emit radiation differing in their spectral distribution, intensity, etc., and the different source-detector systems may be positioned in the same plane or may have an offset along the z-axis 18.

A support 26 supports a subject, such as a human, within the imaging region 22. The support 26 may be movable in order to guide the subject to a suitable location within the imaging region 22 before, during and/or after performing a helical, axial, and/or other scan, for example, by moving the support 26 along the z-axis 18 and/or one or more other axes.

A control component 28 controls each of the x-ray sources 14, including turning the x-ray sources 14 “on” and “off” to commence and terminate the emission of radiation and governing the output of each of the x-ray sources 14. In one instance, at least one of the x-ray sources 14 is driven to continuously emit radiation during a data acquisition cycle. The set of detectors 24 corresponding to the at least one x-ray source 14 detects the radiation that traverses the imaging region 22. The detected radiation is used to generate corresponding signals that can be reconstructed to generate images of a subject residing with the imaging region 22.

With cardiac CT applications, the detected radiation and generated signals provide information about the cardiac cycle. Such data can be used to generate one or more images corresponding to one or more cardiac phases. For example, the data can be used to generate a three-dimensional image for each cardiac phase. In another instance, a series of images representing the different cardiac phases can be viewed as a function of time to create four-dimensional information over the cardiac cycle. Lower resolution images are suitable when using such images to observe the dynamics of heart muscle during a cardiac cycle. As a result, x-ray source power can be reduced during the continuous scan, which reduces patient dose. The power can be set so that the resulting data still provides suitable temporal, spatial and contrast resolution to enable a clinician to view structure of interest. The lower resolution data can be reconstructed to generate lower resolution images, including images of the individual cardiac phases and/or the four-dimensional information.

While driving at least one of the x-ray sources 14 to continuously emit radiation as described above, the control component 28 can concurrently drive at least one other of the at least one x-ray sources 14 to periodically radiation during one or more sampling periods of the same data acquisition cycle. Likewise, the set of detectors 24 corresponding to the at least one x-ray sources 14 detects emitted projection radiation that traverses the imaging region 22, and the detected data is used to generate corresponding signals that can be reconstructed to generate images of a subject residing with the imaging region 22.

For cardiac CT applications, the at least one periodically emitting x-ray sources 14 can be selectively turned “on” to emit radiation during one or more sampling intervals to capture information corresponding to a window around a cardiac phase of interest, and turned “off” otherwise. The resulting signal indicative of the detected radiation is reconstructed and used to generate images of a scanned cardiac phase. In some instances, clinicians prefer detailed images of the individual cardiac phases. For example, coronary artery imaging procedures typically are performed using a higher resolution technique. In such instance, the periodically emitting x-ray sources 14 can be driven in a higher resolution mode than the continuously driven x-ray sources 14. Even though a higher resolution technique is used, patient dose may still be reduced (relative to a continuously drive x-ray source) since the at least one x-ray sources 14 are turned “off” when they are outside of the cardiac window. The resultant data includes higher resolution data that can be reconstructed to generate higher resolution images of the scanned cardiac phase.

By controlling the x-ray sources 14 such that at least one of the x-ray sources 14 continuously emits radiation during a data acquisition cycle and another of the x-ray sources 14 periodically emits radiation during the same data acquisition cycle, the x-ray sources 14 simultaneously emit radiation at least during a portion of the data acquisition cycle. During the periods of simultaneous radiation emission, the sets of detectors 24 for each of the x-ray sources 14 concurrently detect projection data. As a result, the projection data from the continuous and periodic scans can be combined. This includes combining the lower and higher resolution data discussed above. As a result, the temporal, spatial and contrast resolution can be improved and scan time can be reduced. By way of example, if two of the x-ray sources 14 are angularly offset from each other by about 90 degrees relative to the axial 16 and orthogonal to the z-axis 18, then the projection data for the at least two sources 14 can be combined to form a data set for reconstruction (e.g., a 180 degree reconstruction) in less time than it would take to acquire the same data with a single x-ray source system.

In one instance, the output of the x-ray source 14 continuously emitting radiation is controlled (e.g., dose modulated) such that its output changes during the data acquisition cycle. For example, the power of the x-ray source 14 can be increased or decreased depending on the sampling frame. During sampling intervals in which data is collected solely for the lower resolution images, x-ray source power can be reduced to a suitable level as discussed above. However, during sampling intervals in which data is collected for the lower resolution and the higher resolution images, x-ray source power of the at least one x-ray sources 14 continuously emitting radiation can be increased. This includes increasing power to about the same power as the other x-ray source 14. As a result, higher resolution data can be acquired via both the continuously and the periodically driven x-ray sources 14. Combing the projection data from these x-ray sources 14 can further improve temporal, spatial and contrast resolution.

Various techniques can be used to gate the x-ray sources 14 that periodically emit radiation. For instance, the x-ray sources 14 can be gated via prospective gating 32, retrospective gating 34, or kymogram gating 36. With the prospective gating 32 approach, heart electrical activity is concurrently monitored via an ECG device 38 during the imaging procedure. The control component 28 or other component can monitor the electrical activity and upon sensing a landmark within the electrical activity, such as a peak of an R wave, gate the periodically emitting x-ray sources 14 to emit radiation for a sampling period. With the retrospective gating 36 approach, an initial scan (e.g., a pre-scan) is performed along with recording an ECG, and cardiac phases of interest are identified in the resulting images. This can be achieved through the lower resolution images to reduce patient dose. This data is used during subsequent scanning to gate the periodically driven x-ray source 14 during a cardiac CT procedure. Alternatively, images reconstructed during the procedure corresponding to the continuously driven x-ray sources 14 are used to identify desired cardiac phases and gate the periodically emitting x-ray sources 14. With kymogram gating 36, raw projection data is analyzed. For example, a trajectory of the center of the mass of a beating heart is determined from the raw data and analyzed to determine and/or locate cardiac phases. The moment of the center of mass can be calculated and monitored for changes that are indicative of the different cardiac phases.

When the concurrently and periodically driven x-rays sources 14 are emitting radiation, each of the x-ray sources 14 simultaneously emits radiation through the imaging region 22. As a result, each detector in each set of detectors 24 detects primary radiation emitted by a corresponding one of the x-ray sources 14 and cross scatter radiation from the other x-ray sources 14. By additionally detecting only cross scatter radiation (no primary radiation) at each detector, a scatter correction signal can be generated for each detector. The scatter correction signal can be used to scatter correct the projection to substantially remove the cross scatter components from the projection data.

With the periodically emitting x-ray sources 14, a corresponding set of detectors 24 can be activated when the x-ray sources 14 are not emitting radiation in order to detect cross radiation from the other x-ray sources 14. Such radiation can be detected throughout at least a portion of time the x-ray sources 14 are not emitting radiation. This interval can be determined in connection with the x-ray source gating approaches (e.g., prospective, retrospective, and kymography) discussed above and/or other techniques. The sampling of the cross scatter radiation during this interval can be variously determined. For example, it can be based on an angular rate at which the cross scatter radiation changes over the angle of rotation of the x-ray sources 14 about the imaging region 22. For frames in which cross scatter is not sampled, the acquired samples can be used to derive the samples. For instance, an interpolation or other technique can be used to generate the samples. The detected and/or derived samples can then be used to create scatter correction signal for scatter correcting the projection data.

A similar technique can be used in connection with the continuously driven x-ray sources 14. For instance, the continuously driven x-ray sources 14 can be turned “off” for a cross scatter sampling period when the periodically driven x-ray sources 14 are emitting radiation. The corresponding set of detectors 24 can be activated to detect cross scatter radiation from these x-ray sources 14. Likewise, the acquired samples can be used to derive additional samples and form scatter correction data. If desired, cross scatter radiation can also detected during the periods in which the periodically emitting x-ray sources 14 are not emitting radiation. For instance, during these periods, the continuously driven x-ray sources 14 can be turned “off” and the periodically emitting x-ray sources 14 can be turned “on” for a cross scatter sampling period. Again, the set of detectors 24 corresponding to the continuously driven x-ray sources 14 can detect cross scatter radiation from the periodically driven x-ray sources 14.

The sampling of the cross scatter radiation can be based the desired resolution of the lower resolution images, the cross scatter angular frequency, statistics, the quality of the scatter correction, etc. In one instance, only the projection data corresponding to the higher resolution images is scatter corrected; the projection data used to generate the lower resolution images are not scatter corrected, for example, in instances in which the resulting images are suitable to the clinician without scatter correction. These samples can also be used to derive additional samples and form the scatter correction data.

The data from the both the continuously and the periodically driven x-ray sources 14 is conveyed to a reconstruction system 40 that reconstructs the signals to generate volumetric data indicative of the scanned region of the subject. An image processor 42 processes the volumetric image data generated by the reconstruction system 40. As discussed above, this can include generating lower and/or higher resolution images (e.g., 3D and 4D) of the cardiac cycle and/or one or more desired cardiac phases. The generated images can then be displayed, filmed, archived, forwarded to a treating clinician (e.g., emailed, etc.), fused with images from other imaging modalities, further processed (e.g., via measurement and/or visualization utilities and/or a dedicated visualization system), stored, etc.

A computing system (or console) 44 facilitates operator interaction with and/or control of the scanner 12. Software applications executed by the computing system 46 allow the operator to configure and/or control operation of the scanner 12. For instance, the operator can interact with the computing system 44 to select scan protocols, initiate, pause and terminate scanning, view images, manipulating volumetric image data, measure various characteristics of the data (e.g., CT number, noise, etc.), etc. The computing system 44 communicates various information to the control component 28, including, but not limited to, instructions and/or parameters such as x-ray source resolution, gating approach, x-ray source power, data combining scheme, cross scatter correction technique, etc. The control component 28 uses such information as described above to control the scanner 12.

FIG. 2 illustrates an exemplary gating technique in which the periodically emitting x-ray sources 14 are gated via an ECG signal. For sake of brevity and clarity only two of the x-ray sources 14 are illustrated. In this non-limiting example, the x-ray source 14, is “on” during each data acquisition cycle such that it continuously emits radiation during the data acquisition cycles. This is illustrated by a driving signal 46 that is continuously “on” during data acquisition. The x-ray source 14_{N} periodically emits radiation during each data acquisition cycle.

The periodically emitting x-ray source 14_{N} is gated to an ECG signal 48 that is acquired while performing the CT procedure. Desired cardiac phases 50 and 52 are identified within the ECC signal 48. A characteristic of the ECG signal 48 is used to trigger the gating of the periodically emitting x-ray source 14_{N}. For example, a peak of an R wave 54 of the ECG 48 can be used to gate the periodically emitting x-ray source 14_{N} in connection with the cardiac phase 50, and a peak of an R wave 56 of the ECG 48 can be used to gate the periodically emitting x-ray source 14_{N} in connection with the cardiac phase 52.

Upon sensing the peaks 54, 56, the periodically emitting x-ray source 14_{N} can be activated (immediately or within a time delay) to begin emitting radiation. After a lapse of a time period or completion of an angular movement, the periodically emitting x-ray source 14_{N} is turned “off.” In this example, the periodically emitting x-ray source 14_{N} is activated to emit radiation during the desired cardiac phases 50 and 52. This is illustrated by the signal 58, which is in “on” states 60 and 62 during the cardiac phases 50 and 52, respectively, and in “off” states 64, 66, 68 outside of the cardiac phases 50 and 52.

FIG. 3 illustrates a non-limiting method for gating the x-ray sources 14 of the multi-source medical imaging system 10. At reference numeral 70, the control component 28 controls the at least two x-ray sources 14 such that at least one of the x-ray sources 14 continuously emits radiation during a data acquisition cycle. This radiation can be used to generate various images such as one or more three-dimensional images of the cardiac phases, a series of images representing the different cardiac phases as a function of time, etc. Such images can be used to observe the dynamics of heart muscle over a cardiac cycle. Since low resolution images are suitable for such images, x-ray source power can be reduced during the continuous scan, which can reduce patient dose.

At 72, the control component 28 concurrently controls at least one other of the x-ray sources 14 to periodically emit radiation during one or more sampling intervals (e.g., a desired cardiac phase) of the data acquisition cycle. This can be achieved by gating the periodically emitting x-ray sources 14 with a suitable gating mechanism, including, but not limited to, the prospective gating 32, the retrospective gating 34, and the kymogram gating 36 techniques. The detected data can be used to generate detailed images of a scanned cardiac phase. In such instance, the periodically emitting x-ray sources 14 can be driven in a higher resolution mode relative to the continuously driven x-ray sources 14 to produce higher resolution images.

At 74, projection data corresponding to the continuously driven is detected with corresponding detectors from the sets of the detectors 24, and projection data corresponding to the periodically driven x-ray sources 14 is detected with corresponding detectors from the sets of the detectors 24. In one instance, the projection data is scatter corrected since the data includes cross scatter radiation. Scatter correction signals can be obtained by detecting only cross scatter during cross scatter sampling periods in which only one of the x-ray sources 14 is emitting radiation as discussed above. The projection data is then used to generate signals indicative of the detected radiation. This is done for both the projection data corresponding to the continuously driven x-ray sources 14 and the projection data corresponding to the periodically driven x-ray sources 14.

At 76, both sets of the projection data can be conveyed to the reconstruction system 40 and reconstructed to generate one or more images. As discussed above, this can include generating detailed higher resolution images of a desired cardiac phase generated with data corresponding with the periodically emitting x-ray source 14, and lower resolution images, including 4D images, generated with data corresponding with the continuously emitting x-ray source 14. In addition, the projection data associated with the continuously and periodically emitting x-ray sources 14 can be combined to generate data that can be used to further improve image resolution. Moreover, the output of the continuously driven x-ray source 14 can be modulated to increase the resolution of the data associated therewith when emitting radiation concurrently with the periodically driven x-ray sources 14. Combining such data can further improve the resolution of the images.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A computed tomography system comprising:
at least a first x-ray source that continuously emits radiation through an imaging region while rotating about the imaging region during a data acquisition cycle;
at least a second x-ray source that periodically emits radiation through the imaging region while rotating about the imaging region during the data acquisition cycle;
a first set of detectors that detects projection radiation corresponding to the at least first x-ray source and generates first projection data indicative of the detected radiation;
a second set of detectors that detects projection radiation corresponding to the at least second x-ray source and generates second projection data indicative of the detected radiation; and
a reconstruction system that reconstructs at least one of the first projection data to generate a set of images, the second projection data to generate a set of images, and a combination of the first and second projection data to generate another set of images.

2. The system of claim 1, wherein the at least second x-ray source is gated to emit radiation during select data acquisition sampling intervals of the data acquisition cycle.

3. The system of claim 2, wherein the gating techniques includes at least one of a prospective gating, a retrospective gating, a ECG gating and a kymogram gating technique.

4. The system of claim 1, wherein the at least second x-ray source is activated to emit radiation during a cardiac phase of interest.

5. The system of claim 1, wherein the at least first and the at least second x-ray sources emit radiation indicative of different resolution.

6. The system of claim 1, wherein the at least first x-ray source emits first resolution radiation and the at least second x-ray source emits second resolution radiation, wherein the second resolution radiation is representative of higher resolution radiation than the first resolution radiation.

7. The system of claim 6, wherein the first resolution radiation and the second resolution radiation are one of equal and different.

8. The system of claim 1 wherein the radiation emitted by the at least first x-ray source is modulated to produce lower resolution data when the at least second x-ray source is not emitting radiation and higher resolution data when the at least second x-ray source is emitting radiation.

9. The system of claim 1, wherein a portion of the first projection data is combined with the second projection data and the combined projection data is used to generate a set of images with a higher resolution than the sets of images generated with the first and the second projection data.

10. The system of claim 1, wherein the reconstructed images include one or more of a higher resolution image of a cardiac phase and a series of three-dimensional images as a function of time.

11. The system of claim 1, wherein the second set of detectors detects cross scatter radiation from the first x-ray source when the second x-ray source is not emitting radiation and the cross scatter radiation is used to scatter correct the second projection data.

12. The system of claim 1, wherein the first set of detectors detects cross scatter radiation from the second x-ray source when the first x-ray source is not emitting radiation and the cross scatter radiation is used to scatter correct the first projection data.

13. A computed tomography x-ray source control method comprising:
continuously emitting radiation through an imaging region with a first x-ray source during a data acquisition cycle;
periodically emitting radiation through the imaging region with a second x-ray source during one or more sampling intervals of the data acquisition cycle;
detecting first projection radiation corresponding to the first x-ray source;
detecting second projection radiation corresponding to the second x-ray source;
reconstructing at least one of the first, the second, and combination of the first and second projection data to generate one or more sets of corresponding images.

14. The method of claim 13 further including gating the second x-ray source to emit radiation during a desired cardiac phase.

15. The method of claim 13 further including gating the second x-ray source to emit radiation using one of a prospective gating, a retrospective gating, and a kymogram gating.

16. The method of claim 13 wherein the first x-ray source emits first resolution radiation and the second x-ray source emits second resolution radiation and the first resolution radiation is lower resolution than the second resolution radiation.

17. The method of claim 13, wherein the first set of images includes a high resolution image of a cardiac phase.

18. The method of claim 13 wherein the second set of images includes a one of low resolution images of one or more cardiac phase and four-dimensional information.

19. The method of claim 13 further including scatter correcting the second projection data with cross scatter radiation from the first x-ray source.

20. A CT imaging system comprising:
means for continuously emitting radiation through an imaging region with one x-ray source and periodically emitting radiation through the imaging region with a different x-ray source;
means for gating the different x-ray source to control when it emits radiation;
means for detecting radiation associated with the x-ray sources; and
means for reconstructing the detected radiation to generate images.
